(19) United States
(12) Patent Application Publication
(10) Pub. No.: US 2015/0250912 A1
(43) Pub. Date: Sep. 10, 2015

(54) FRAGRANCE FORMULATION FOR SCENT DELIVERY DEVICE

(71) Applicant: INTERNATIONAL FLAVORS & FRAGRANCES INC., New York, NY (US)

(72) Inventors: Joseph Brain, Colts Neck, NJ (US); Bozena Marianna Piatek, Middletown, NJ (US); Emmanuel Manna, Monmouth Junction, NJ (US)

(21) Appl. No.: 14/436,330

(22) PCT Filed: Apr. 25, 2013
(86) PCT No.: PCT/US2013/038117

§ 371 Date: Apr. 16, 2015

Related U.S. Application Data
(60) Provisional application No. 61/639,246, filed on Apr. 27, 2012.

(51) Int. Cl. A61L 9/14; C11B 9/00

(52) U.S. Cl. CPC A61L 9/14; C11B 9/00

(57) ABSTRACT

A liquid fragrance formulation is provided. The fragrance formulation has a viscosity in the range of 0.0 to 9.0 cPa, a calculated water-octanol partition coefficient in the range of −1.0 to 4.0; and a surface tension in the range of 0.0 to 25.0 mN/m and is suitable for use in air fresheners, particularly nebulizing scent delivery devices.

### BACKGROUND OF THE INVENTION

Fragrance delivery systems and methodologies in various forms have been in use for many years. Such systems include candles, heated oils, atomizers, diffuser devices including wick-type diffusers, as well as other fragrance transport mechanisms such as nebulizing or atomizing devices (see, e.g., US 2011/0266359, US 2011/0268605, U.S. Pat. No. 6,793,149 and U.S. Pat. No. 7,070,121). These devices may be used in a number of applications ranging from aromatherapy to environmental odor control.

Various compositions which include fragrance materials are known in the art including U.S. Pat. No. 5,449,512 and U.S. Pat. No. 5,160,494. While various fragrance delivery systems have been developed, the design of fragrances with a quality of spray is needed.

### SUMMARY OF THE INVENTION

This invention is a fragrance formulation for a scent delivery device and a method for providing fragrance to the environment. The fragrance formulation is composed of one or more fragrances and an optional carrier, wherein the formulation has a viscosity in the range of 0.0 to 9.0 cPa, a calculated water-octanol partition coefficient (ClogP) in the range of −1.0 to 4.0; and a surface tension in the range of 0.0 to 25.0 mN/m. In certain embodiments, the scent delivery device is a nebulizing scent delivery device.

### DETAILED DESCRIPTION OF THE INVENTION

It has now been found that the combined parameters of viscosity, ClogP and surface tension are determining factors for the quality of spray delivered by scent delivery device, in particular a fragrance nebulizer. Specifically, it was demonstrated that a very strong plume is achieved when a liquid fragrance formulation has a viscosity in the range of 0.0 to 9.0 cPa, a water-octanol partition coefficient (ClogP) in the range of −1.0 to 4.0; and a surface tension in the range of 0.0 to 25.0 mN/m.

For the purposes of this invention, a nebulizing scent delivery device is device having an atomizer to atomize a liquid fragrance oil into a scented mist and deliver the scented mist to air outside of the atomizer. Exemplary nebulizing scent delivery devices of use with the fragrance formulations of the present invention include, but are not limited to, those described in US 2011/0266359 and US 2011/0268605.

Viscosity describes a fluid's internal resistance to flow and may be thought of as a measure of fluid friction. The viscosity of a formulation of the invention can be determined using a viscometer. A viscometer is an instrument that measures the force required to rotate a spindle at a specific rate. There are other types of viscometers as well including some that use objects, such as bubbles or balls, for measuring. In some embodiments of this invention, the viscosity of the fragrance formulation is in the range of 0.0 to 9.0 cPa, e.g., as measured at 25° C. In other embodiments, the viscosity of the fragrance formulation is in the range of 1.0 to 9.0, 2.0 to 9.0, 3.0-9.0, 4.0-9.0, 5.0-9.0, 6.0-9.0, 7.0-9.0, or 8.0-9.0. In yet other embodiments, the viscosity of the fragrance formulation is in the range of 5.1 to 9.0, 5.1 to 8.0, 5.1-7.0, or 5.1-6.0.

As demonstrated herein, a fragrance with a viscosity of greater than 9.0 (e.g., Sweet Dreams, see Example 2) can be decreased by combining said fragrance with a second fragrance or carrier having a viscosity of less than 9.0 (e.g., VANORIS, see Example 2). Similarly, a fragrance with a low viscosity (e.g., VANORIS, see Example 2) can be increased by combining said fragrance with a second fragrance or carrier having a higher viscosity (e.g., ethyl vanillin, see Example 2).

As is conventional in the art, the degree of hydrophobicity of a perfume ingredient can be correlated with its octanol/water partitioning coefficient P. The octanol/water partitioning coefficient of a fragrance ingredient is the ratio between its equilibrium concentration in octanol and in water. A fragrance ingredient with a greater partitioning coefficient P is more hydrophobic. Conversely, a fragrance ingredient with a smaller partitioning coefficient P is more hydrophilic. Since the partitioning coefficients of the perfume ingredients normally have high values, they are more conveniently given in the form of their logarithm to the base 10, logP. In particular embodiments, the logP values are more conveniently calculated by the “CLOGP” program, available from Daylight Chemical Information Systems. The “calculated logP” (ClogP) is determined by the fragment approach of Hansch & Leo (cf., Leo, in Comprehensive Medicinal Chemistry, Vol. 4, Hansch, et al., Eds., p. 295, Pergamon Press, 1990). The fragment approach is based on the chemical structure of each fragrance ingredient, and takes into account the numbers and types of atoms, the atom connectivity, and chemical bonding. The ClogP values, which are the most reliable and widely used estimates for this physicochemical property, are used instead of the experimental logP values in the selection of fragrance ingredients that are useful in this invention. In some embodiments of this invention, the ClogP of the fragrance formulation is in the range of −1.0 to 4.0. In other embodiments, the ClogP of the fragrance formulation is in the range of 0.0 to 4.0, 1.0 to 4.0, 2.0 to 4.0, or 3.0 to 4.0. In yet other embodiments, the ClogP of the fragrance formulation is in the range of 0.0 to 3.0, 1.0 to 3.0, or 2.0 to 3.0.

Non-limiting examples of fragrances that have ClogP values in the range of −1.0 to 4.0 are allyl heptanoate, anethole USP, benzaldehyde, benzyl acetate, cis-3-hexenyl acetate, cis-jasmone, coumarin, dihydromyrcenol, dimethyl benzyl carbinyl acetate, ethyl vanillin, eucalyptol, eugenol, iso eugenol, isobutyl salicylate, flor acetate, geraniol, hydroxycitronellal, koavone, LIFFAROME, dihydro linalool, linalool, methyl anthranilate, methyl beta naphthyl ketone, methyl dihydro jasmonate, nerol, nonalactone, orange flower ether, phenyl ethyl acetate, phenyl ethyl alcohol, phenyl propyl alcohol, phenoxy ethyl isobutyrate, phenoxanol, alpha terpineol, tetrahydro linalool, beta terpineol, vanillin, and mixtures thereof.

As is known in the art, surface tension is a property of the surface of a liquid that allows it to resist an external force. Any conventional method in the art can be used to determine surface tension including, but not limited to, the Du Nouy-Padday method, the spinning drop method, the pendant drop method, the bubble pressure method, the capillary rise method, or the stalagmometric method. Surface tension can be expressed as force per unit length, e.g., milli-Newton per meter (mN/m). In some embodiments of this invention, the surface tension of the fragrance formulation is in the range of 0.0 to 25.0 mN/m. In other embodiments, the surface tension of the fragrance formulation is in the range of 0.0 to 20.0, 0.0 to 15.0, 0.0 to 10.0, or 0.0 to 5.0 mN/m. In yet other embodiments, the surface tension of the fragrance formulation is in the range of 5.0 to 25.0, 10.0 to 25.0, 15.0 to 25.0 or 20.0 to 25.0 mN/m.

Non-limiting examples of fragrances that have a surface tension in the range of 0.0 to 25.0 mN/m are limonene D, VANORIS, orange flower ether, anethole, cis-jasmone, cis-3-hexenyl benzoate, dihydro linalool, and tetrahydro linalool.

In some embodiments, the fragrance formulation is composed of a first fragrance and one or more second fragrances. To achieve the characteristics described herein (i.e., viscosity, ClogP, and surface tension), desirably the first and/or second fragrances exhibit at least one or more of a viscosity in the range of 0.0 to 9.0 cPa, a ClogP in the range of −1.0 to 4.0; or a surface tension in the range of 0.0 to 25.0 mN/m. In this respect, the first fragrance and one or more second fragrances, when combined, provide a fragrance formulation having an overall viscosity in the range of 0.0 to 9.0 cPa, a ClogP in the range of −1.0 to 4.0; and a surface tension in the range of 0.0 to 25.0 mN/m.

In some embodiments, the fragrances are formulated with a carrier or solvent. Carriers of use in this invention include, but are not limited to, alcohols such as ethanol, methanol, and the like; dipropylene glycol, dipropylene glycol ethers, diethyl phthalate and isopropyl myristate. The level of water may be kept to a minimum, and is preferably below 5 weight percent of the fragrance formulation, more preferably below 1 weight percent and most preferably less than 0.1 weight percent. In this respect, the invention includes the preparation of a liquid, non-aqueous fragrance formulation. Persons of skill in the art will be able to prepare fragrance formulations within the scope of the present invention that contain no intentionally added water.

The fragrance formulation of the present invention is well suited for use in air fresheners. In this respect, the fragrance formulation of this invention finds use in a method for providing fragrance to the environment. In accordance with this method, the fragrance formulation described herein is contained within a scent delivery device, e.g., a nebulizing scent delivery device, and delivered to the environment via said device to provide a fragrance.

The invention is described in greater detail by the following non-limiting examples.

### EXAMPLE 1

### Physical Properties of Fragrance Ingredients

Physical properties of ingredients of fragrance formulations and their effect on the performance of a nebulizing device was evaluated visually (intensity of the plume) and instrumentally (Malvern droplet size analyzer). The nebulizing device used in this analysis was that described in WO 2010/079485. This analysis indicated that the combination of three parameters: viscosity, ClogP and surface tension, determined the quality of spray delivered by the nebulizing device (Table 1). Specifically, a viscosity in the range of 0 to 9.0 cPa, a ClogP in the range of −1.0 to 4.0 and a surface tension in the range of 0.0 to 25.0 mN/m provided a formulation with the best plume.

TABLE 1

| | | | | | | | | Almanac | |
|---|---|---|---|---|---|---|---|---|---|
| | | | | | | | | Surface | |
| | | Measured | | V % | | | | Tension | |
| | | Viscosity | | <15 | | Almanac | | (mN/m) | Functional |
| Ingredient | Plume | (cPa | Cv | un | D90 | ClogP | MW | (Measured) | Group |
| Water | 4 | 1.00 | 1.8 | 80 | 19.50 | | | 72.00 | |
| Ethanol | 5 | 1.20 | 1.2 | 84 | 17.00 | | | 22.00 | |
| Limonene D | 5 | 0.89 | 7.65 | 75 | 22.00 | 4.42 | 138 | 21.86 | Terpine |
| LIFFAROME | 5 | 1.36 | 4.97 | 78 | 20.00 | 2.26 | 158 | 25.48 | Ester |
| | | | | | | | | | (1&2alc) |
| Allyl Heptanoate | 5 | 1.37 | 5.6 | 80 | 19.60 | 3.40 | 170 | 28.07 | Ester |
| | | | | | | | | | (1alc) |
| VANORIS | 5 | 1.92 | 4.2 | 88 | 15.87 | 4.08 | 215 | 24.54 | Ester |
| | | | | | | | | (24.10) | (1alc) |
| Dihydro | 5 | 2.18 | 2.5 | 81 | 19.50 | 2.99 | 156 | 21.3 | 3Alc |
| Myrcenol Rect | | | | | | | | | |
| Orange Flower Ether | 5 | 2.19 | 2.9 | 83 | 18.50 | 3.54 | 168 | 22.29 | Ether |
| Benzyl Acetate | 4 | 2.23 | 3.9 | 79 | 20.00 | 1.73 | 150 | 32.25 | Ester |
| Anethole USP | 5 | 2.69 | 2.2 | 75 | 23.00 | 3.39 | 148 | 19 | Ether |
| Cis-Jasmone | 3 | 2.73 | 1.7 | 70 | 30.80. | 2.91 | 164 | 17.64 | Ketone |
| Cis-3-Hexenyl Benzoate | 5 | 3.35 | 2.1 | 80 | 20.00 | 4.25 | 204 | 23.35 | Ester |
| | | | | | | | | (33.10) | (1alc) |
| Isobutyl Salicylate | 5 | 4.22 | 1.3 | 86 | 17.70 | 3.71 | 194 | 33.80 | Ester |
| | | | | | | | | (33.20) | (1alc) |
| Isopropyl Myristate | 2 | 4.83 | 0.6 | 83 | 27.40 | 7.51 | 270 | 31.90 | Ester |
| | | | | | | | | | (2alc) |
| Isobornyl Propionate | 2 | 5.40 | 0.3 | 82 | 22.00 | 4.49 | 210 | 29.4 | Ester |
| | | | | | | | | | (2alc) |
| VERDOX | 2 | 5.63 | 0.5 | 79 | 27.00 | 4.02 | 198 | 25.18 | Ester |
| | | | | | | | | | (2alc) |
| Phenoxy Ethyl Isobutyrate | 2 | 5.67 | 0.4 | 84 | 17.00 | 2.94 | 208 | 29.02 | Ester |
| | | | | | | | | | (1alc) |
| | | | | | | | | | Ether |
| Dihydro Linalool | 3 | 8.06 | 0.9 | 84 | 19.50 | 2.98 | 156 | 20.22 | 3Alc |
| | | | | | | | | (26.50) | |
| Phenyl Propyl Alcohol | 1 | 15.00 | | | | 1.89 | 136 | 35.26 | 1Alc |
| | | | | | | | | (37.10) | |
| Tetrahydro Linalool | 2 | 15.20 | 0.2 | 81 | 24.00 | 3.54 | 158 | 21.60 | 3Alc |
| | | | | | | | | (24.10) | |
| Phenoxanol | 1 | 28.00 | | | | 3.38 | 178 | 33.3 | 1Alc |
| Di-Propylene Glycol | 1 | 101.00 | | | | −2.00 | 134 | 27.10 | 1Alc Ether |
| Delta | 2 | 7.65 | | | | | | | |
| Decalactone | | | | | | | | | |
| Vertenex | 2 | 7.45 | | | | | | | |
| Geraniol 980 | 2 | 7.94 | | | | | | | |
| Methyl DH Jasmonate | 1 | 11.40 | | | | | | | |
| Iso Bornyl Acetate | 2 | 7.76 | | | | | | | |
| Iso E Super | 1 | 37.10 | | | | | | | |
| Veridian | 5 | 1.98 | | | | | | | |
| Undecavertol | 2 | 12.6 | | | | | | | |
| Montaverdi | 5 | 1.82 | | | | | | | |
| Galbascone BHT | 4 | 4.47 | | | | | | | |

5, very good/strong plume;
4, good plume;
3, OK/poor plume;
2, very poor plume;
1, no plume.

### EXAMPLE 2

### Measured Viscosity of Fragrance Ingredients

The viscosity of various fragrance formulations was determined. The results of this analysis are presented in Table 2.

TABLE 2

| Ingredients | Measured Viscosity |
|---|---|
| 5% Ethyl Vanillin in VANORIS | 1.93 |
| 10% Ethyl Vanillin in VANORIS | 1.98 |
| 20% Ethyl Vanillin in VANORIS | 2.01 |
| Sweet Dreams | 18.40 |
| Sweet Dreams + 10% VANORIS | 12.40 |
| Sweet Dreams + 20% VANORIS | 8.87 |
| Sweet Dreams + 50% VANORIS | 4.44 |

What is claimed is:

1. A fragrance formulation for a scent delivery device comprising one or more fragrances and an optional carrier, wherein the formulation has a viscosity in the range of 0.0 to 9.0 cPa, a calculated water-octanol partition coefficient (ClogP) in the range of −1.0 to 4.0; and a surface tension in the range of 0.0 to 25.0 mN/m.

2. The fragrance formulation of claim 1, wherein the scent delivery device is a nebulizing scent delivery device.

3. A method for providing fragrance to the environment comprising providing the fragrance formulation of claim 1 to a scent delivery device.

4. The method of claim 3, wherein the scent delivery device is a nebulizing scent delivery device.

5. A scent delivery device that contains the fragrance formulation of claim 1.

6. The scent delivery device of claim 5, wherein the device is a nebulizing scent delivery device.
